(19) United States
(12) Patent Application Publication
(10) Pub. No.: US 2026/0096549 A1
(43) Pub. Date: Apr. 9, 2026

(54) DRONE BASED PRECISION AGRICULTURE FIELD MANAGEMENT SYSTEM

(71) Applicant: Microchip Technology Incorporated, Chandler, AZ (US)

(72) Inventors: Patrick McFarland, Gilbert, AZ (US); Bomy Chen, Newark, CA (US); Arthur B. Eck, Gilbert, AZ (US); Steve Nagel, Chandler, AZ (US)

(73) Assignee: Microchip Technology Incorporated, Chandler, AZ (US)

(21) Appl. No.: 18/938,386

(22) Filed: Nov. 6, 2024

Related U.S. Application Data
(60) Provisional application No. 63/705,437, filed on Oct. 9, 2024.

(51) Int. Cl. A01M 7/00; A01C 21/00; A01C 23/04; B64U 20/87; B64U 80/30; B64U 101/40; B64U 101/45; G01N 33/00; G01N 33/24; G05D 1/689; G05D 105/80; G05D 107/20

(52) U.S. Cl. CPC A01M 7/0089; A01C 21/007; A01C 23/04; B64U 20/87; B64U 80/30; G01N 33/0098; G01N 33/245; G05D 1/689; B64U 2101/40; B64U 2101/45; B64U 2201/10; G05D 2105/87; G05D 2107/21

(57) ABSTRACT

Systems and methods for imaging a crop field, probing soil of a location of the field based on the imaging of the field to obtain diagnostic data, sampling soil of a location of the field based on the imaging of the field; and spraying the field based on the imaging of the field, the probing soil, or the sampling soil. A system uses a tractor having a spray system, a drone having a tool deployment module comprising a reel and a line, a drone launch pad comprising an imaging module attachable to and detachable from the line, a probing module attachable to and detachable from the line, a sample collection module attachable to and detachable from the line, and drone batteries, a controller to receive diagnostic data from the imaging module, the probing module, or the sample collection module, and to transmit spray instructions to the spray system.

### RELATED PATENT APPLICATION

This application claims priority to commonly owned U.S. patent application Ser. No. 63/705,437 filed Oct. 9, 2024, the entire contents of which are hereby incorporated by reference for all purposes.

### TECHNICAL FIELD

The present disclosure relates to agriculture, in particular, artificial intelligence informed field spraying based on crop yield diagnostic data from the field.

### BACKGROUND

Drones are increasingly being used in agriculture to monitor crop health but face several challenges that prevent their adoption. They have limited battery life which is impractical for covering hundreds or thousands of acres quickly. Drones typically use a spectral/camera-based system and an image-based artificial intelligence (AI) to diagnose issues such as pests, disease, or nutrient deficiency, which suffers from false positives/false negatives. When an AI diagnosed issue is manually verified by a person in the field, the AI model receives no feedback on verification, whether the AI's inference is confirmed or rejected. Additionally, when testing, such as soil testing is performed, it is expensive and has significant latency due to occurring several months after the issue arises and does not show individualized data for each plant but rather an entire region. Because of this, soil testing regimes are not very precise due to a small number of samples over a large area which might not represent issues each plant may have. Drones have been employed to attempt to address labor shortages and labor costs in agriculture but have fallen short for the formerly described reasons. A need remains to reduce human man-hours, verification, and costs.

Current approaches to this include having the drone carry all of the equipment, including liquid tanks for things like pesticides. This is impractical as it requires a large expensive drone, short battery life, and human involvement to collect the drone.

The known approach to image-based AI is to gather large amounts of data and feed it into training where a model is eventually produced. A drawback to this approach is that there are limits to the amount of data available, such as a finite number of images to use, as well as the fact that after a certain point, the AI has learned all it can from the majority of the data. Most of the data being fed at that point is “junk data” and wastes precious computer capacity including time, energy, and costs without limitation.

There is a need for systems and methods to provide “nutritious data” to artificial intelligence engines to inform field spray.

### SUMMARY OF THE INVENTION

Aspects provide a system comprising: a drone having a data collection module to collect crop yield factor data from a crop field; a sprayer to spray a condition treating fluid; a drone station to receive crop yield factor data from the data collection module of the drone, and comprising an artificial intelligence crop field model circuit configured to: diagnose crop conditions based on the crop yield factor data; and instruct the sprayer to spray the condition treating fluid on a portion of the crop field based on diagnosed crop conditions.

An aspect of the system as in the preceding paragraph provides a system, wherein the data collection module comprises an image sensing device to collect crop yield factor data comprising: ultraviolet (UV) images, visible spectrum (VIS) images, near-infrared (NIR) images, short-wave infrared (SWIR) images, thermal images, light detection and ranging (LIDAR) images, radio detection and ranging (RADAR) images, or sound navigation and ranging (SONAR) images.

An aspect of the system as in one of the preceding two paragraphs provides a system, wherein the data collection module comprises a soil probe to collect crop yield factor data comprising: level of nitrogen in the soil, level of phosphorus in the soil, level of potassium in the soil, soil temperature, pH level of soil, electrical conductivity of the soil, or soil humidity or moisture level.

An aspect of the system as in one of the preceding three paragraphs provides a system, wherein the data collection module comprises a soil sampler to collect crop yield factor data comprising: pH of the soil, soil lime content, soil phosphorus content, soil potassium content, soil calcium content, soil magnesium content, soil zinc content, soil manganese content, soil cation exchange capacity, or soil microbial activity, or soil microbiome.

An aspect of the system as in one of the preceding four paragraphs provides a system, comprising a reel connected to the drone and a line wound on the reel, wherein one end of the line is releasably connected to the data collection module, wherein the reel and line are configured to deploy and retrieve the data collection module relative to the drone.

An aspect of the system as in one of the preceding five paragraphs provides a system, wherein the sprayer is connectable to a tractor.

An aspect of the system as in one of the preceding six paragraphs provides a system, wherein the drone station comprises: a battery charger to receive and charge a drone battery; and a parking space to park an image sensing device, a soil probe, or a soil sampler.

An aspect of the system as in one of the preceding seven paragraphs provides a system, wherein the drone station is associated with a tractor.

An aspect of the system as in one of the preceding eight paragraphs provides a system, wherein the drone station comprises a soil diagnostic laboratory to analyze soil samples.

An aspect provides a method comprising: collecting crop yield factor data from a crop field via a data collection module transported by a drone; generating an artificial intelligence crop field model based on collected crop yield factor data; diagnosing crop conditions based on the artificial intelligence crop field model; and instructing a sprayer to spray condition treating fluid on a portion of the crop field based on diagnosed crop conditions.

An aspect of the method as in the preceding paragraph provides a method, wherein collecting crop yield factor data comprises collecting data via an image sensing device, the crop yield factor data comprising: ultraviolet (UV) images, visible spectrum (VIS) images, near-infrared (NIR) images, short-wave infrared (SWIR) images, thermal images, light detection and ranging (LIDAR) images, radio detection and ranging (RADAR) images, or sound navigation and ranging (SONAR) images.

An aspect of the method as in one of the preceding two paragraphs provides a method, wherein collecting crop yield factor data comprises collecting data via a soil probe, the crop yield factor data comprising: level of nitrogen in the soil, level of phosphorus in the soil, level of potassium in the soil, soil temperature, pH level of soil, electrical conductivity of the soil, or soil humidity or moisture level.

An aspect of the method as in one of the preceding three paragraphs provides a method, wherein collecting crop yield factor data comprises collecting data via a soil sampler, the crop yield factor data comprising: pH of the soil, soil lime content, soil phosphorus content, soil potassium content, soil calcium content, soil magnesium content, soil zinc content, soil manganese content, soil cation exchange capacity, or soil microbial activity, or soil microbiome.

An aspect of the method as in one of the preceding four paragraphs provides a method, comprising: deploying the data collection module from the drone; collecting crop yield factor data via the data collection module; and retrieving the data collection module to the drone.

An aspect of the method as in one of the preceding five paragraphs provides a method, comprising: charging a drone battery via a battery charger of a drone station; and parking the data collection module in a parking space of a drone station.

An aspect of the method as in one of the preceding six paragraphs provides a method, comprising: transmitting data from the drone to the base station wirelessly or via memory device.

An aspect of the method as in one of the preceding seven paragraphs provides a method, comprising: identifying global navigation satellite system coordinates of the drone station; identifying global navigation satellite system coordinates of a data collection position within the crop field; and navigating the drone from the drone station to the data collection position via the global navigation satellite system.

An aspect of the method as in one of the preceding eight paragraphs provides a method, wherein generating an artificial intelligence crop field model comprises: creating a crop field map with global navigation satellite system coordinates of individual crop plants; and associating diagnosed crop conditions of individual crop plants in the crop field map.

An aspect of the method as in one of the preceding two paragraphs provides a method, wherein generating an artificial intelligence crop field model comprises: time stamping diagnosed crop conditions.

An aspect of the method as in one of the preceding two paragraphs provides a method, wherein generating an artificial intelligence crop field model comprises: training the AI model with the crop yield factor data in at least near real-time.

An aspect of the method as in one of the preceding two paragraphs provides a method, wherein generating an artificial intelligence crop field model comprises: training the AI model with the crop yield factor data that corrects previous crop yield factor data.

### BRIEF DESCRIPTION OF THE DRAWINGS

The figures illustrate examples of systems and methods that provide “nutritious data” to artificial intelligence engines to inform field spray.

FIG. 1 shows a top view of a crop field being worked by drones and a tractor with spray equipment.

FIG. 2 shows a perspective view of a drone equipped with a camera.

FIG. 3 shows a perspective view of a drone equipped with a line that is retractable to suspend and deliver tools.

FIG. 4 shows a drone equipped with a soil test probe suspended by a retractable line.

FIG. 5 shows a drone equipped with a soil sample probe suspended by a retractable line.

FIG. 6 shows a drone equipped with a soil sample vacuum suspended by a retractable line.

FIG. 7 shows a top view of a drone launch pad.

FIG. 8 shows a block diagram of a crop field diagnostic system. The system comprises a drone launch pad, a tractor spray controller, a soil sample receptacle, a drone, and a probe.

FIG. 9 shows a block diagram of a system to provide “nutritious data” to artificial intelligence engines to inform crop field spray.

FIG. 10 shows a flow chart of a method to provide “nutritious data” to artificial intelligence engines to inform crop field spray.

The reference number for any illustrated element that appears in multiple different figures has the same meaning across the multiple figures, and the mention or discussion herein of any illustrated element in the context of any particular figure also applies to each other figure, if any, in which that same illustrated element is shown.

### DESCRIPTION

According to an aspect, there is provided processes and systems to manage drone and automated agricultural equipment. These processes and technologies enable drones to be lightweight and stay continuously charged for non-stop operation over a large field. They also allow for the automated collection of plant health data so that the tractor can be programmed to dispense the exact nutrient or pesticide solution appropriate for each specific plant. The plant health data can also be provided to the plant grower as a dashboard for the identification of systemic issues. The tractor and drones can be automated so as to minimize the need for human intervention/labor. These technologies enable drones to fly over the field ahead of the tractor with an imaging system that looks for issues affecting crop health, such as pests, hydration, or nutrient deficiencies using an image-based AI. The drone can then fly back to the tractor, pick up a plant test kit that has a fresh battery, fly to the plants of interest and then take a soil sample. This sample can then be tested by the drone using a soil test kit either on the drone or the tractor. Once the issue is verified by a soil test, the tractor can be programmed to dispense a specific solution for those specific plants to treat the issue, as the exact location of the plant in the field is known. For example, this could be an extra dose of a nutrient and a specific pesticide. If the soil test kit shows the image AI produced a false positive or incorrectly labeled the issue, feedback can automatically be sent to the AI model for retraining with the new information to improve the future performance of the model. For crop health issues that can't be identified with a basic test kit carried on the tractor, a central automated warehouse nearby, such as one in the nearest city, could contain the other needed test kits, and even treatments for less frequently encountered issues. The drone could send a request for a more specific test kit.

By automating the crop monitoring process, and by performing image-based screening and then soil-sample verification, truly precision agriculture can be enabled. Dosages can be specific for an individual plant and can be customized based on the soil sample screening. The soil samples don't need to be sent back to a lab to be manually processed where it can take days, weeks or months, it is processed in a few minutes/hours. This enables a significant reduction in costs per test by removing the need to collect, ship, and analyze the samples.

In addition, instead of sending the AI model large amounts of data that it already knows, the AI model can only be sent data that it doesn't know, which would include samples of false positives and false negatives. This greatly cuts down on training time, costs, and energy usage and speeds up and enables more accurate image-based issue classifications in the future.

By feeding only “nutritious data” to the AI, for example data that have been verified that the AI doesn't know, training time, energy and costs can be significantly reduced. Verified data is hard and expensive to obtain, and this solution automates the collection and processing of this verified, “nutritious data. ” The AI generator may generate incorrect models based on incorrect information. For example, image crop yield factor data may be inconclusive and cause the AI model to make incorrect assumptions about plant health or soil conditions. Physical crop yield factor data, such as test results from soil or plant samples, may provide more conclusive data that the AI generator may use to revise its AI model to more correctly represent actual conditions. The AI generator may train the AI model with the crop yield factor data that corrects previous crop yield factor data. This enables customers to get newer, better AI models for use in the fields faster. By using modular drone kits with freshly charged batteries, by using the tractor as a landing pad, and by reducing the kit weights, continuous operation of the drones is enabled. These systems and methods may allow the precise dispensing of nutrients through built-in sprayers on the tractor to each plant, allowing for better crop yields, more revenue, and fewer losses.

According to aspects, the AI imaging model receives instant feedback based on soil test data. This information can then be fed to the cloud for retraining and update. The most important data AI models can be fed are false positives and false negatives. This is the information the AI was wrong about. The AI doesn't need additional “junk” data that it already knows. Feeding the AI things it doesn't know (FP/FN) is “nutritious” data. This “nutritious” data improves the performance of the imaging model. Some data cannot easily be inferred from imaging. Soil testing may be an ongoing and real-time part of the process.

By having the drone be modular and having separate kits with freshly charged batteries, drone weight can be significantly reduced and flying time can be nearly continuous throughout the day. By having a central automated warehouse nearby, less frequently encountered tests or issues can be stored there, rather than needing to store in a test kit or on the tractor, reducing weight, bulk and cost. Because all of these processes are fully automated, the need for human involvement is significantly reduced which represents costs savings, less need for labor intensive activities and the number of required farm staff.

The technologies being proposed include a modified agricultural tractor with a roof that has a drone launch pad. This launch pad holds modular kits that include a battery and either an imaging package or a plant testing package that can be picked up and dropped off by the drones. The launch pad also has replacement back-up batteries that the drones use for power when not connected to a modular kit. When a drone has a low battery, it may fly back to the launch pad and swap out its depleted battery for a charged battery and immediately resume operations. These replacement batteries may constantly be charged by the tractor to allow for continuous operation by the drones. A drone may have two battery ports, so that during flying operations, the drone has one battery in one of its battery ports. When the battery is depleted, it may fly back to the launch pad and land on a battery charger. The battery charger may similarly have two battery ports, with one battery in one of the ports. When the drone lands on the battery charger, the depleted battery is released and deposited in the empty port of the battery charger, and the charged battery in the battery charger is immediately plugged into the empty port of the drone. The drone may have an on-board battery that allows it to maintain power to its electronic control systems during the swap of main power batteries.

The imaging kits include a camera capable of imaging the fields and which image feed is analyzed using an AI model either on the drone, through a data link to the tractor which is hosting an inference server, or connected via the tractor to the cloud. The imaging system can be hyper/multispectral or focused on a few wavelengths of interest, like visible and infrared (IR). The plant testing package is a kit that the drone carries that can take soil samples near the plants of interest. The soil samples can be obtained through direct means such as an auger or soil probe carried in the kit. It could also be obtained via an air pump on the drone that sucks in dust near the plant. To save weight, the soil testing lab can be on the tractor rather than the drone, where the drone only carries the collection equipment.

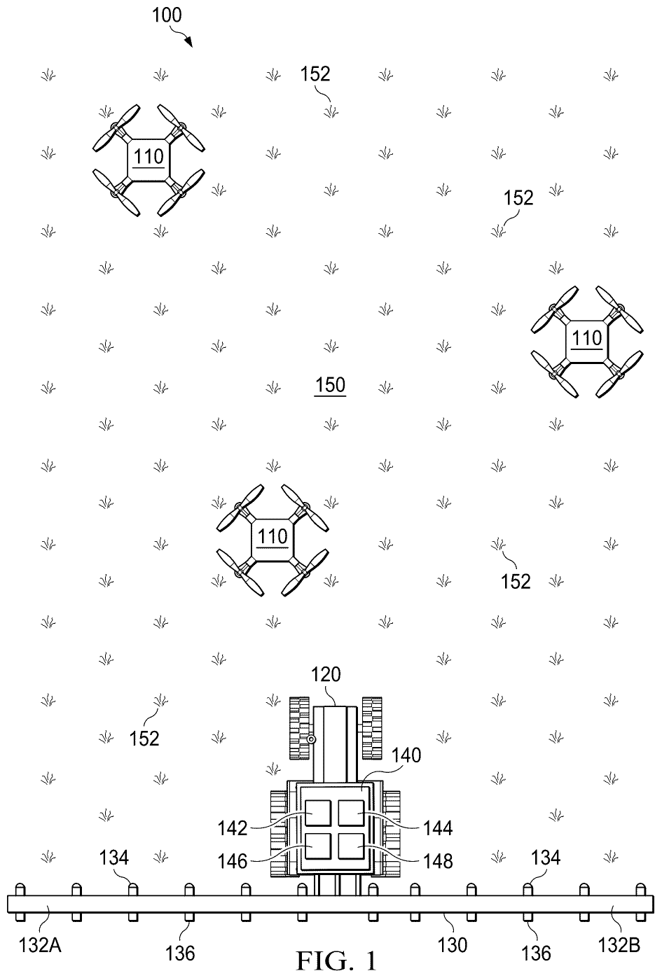

FIG. 1 shows a top view of a crop field 150 being worked by a system 100 comprising drones 110 and a tractor 120 with spray equipment 130. In particular, the tractor 120 is performing an in-season spraying operation, wherein the crop 152 has already been planted in rows in the soil of the crop field 150 and the plants are partially grown. Undesirable plants, such as weeds, may also be growing in the field with the crop 152. The tractor 120 is driving across the crop field 150 with its tires positioned between crop rows. The spray equipment 130 attached to the tractor 120 comprises two long booms 132A and 132B extending in opposite directions from the tractor 120 over the tops of the crop 152. The spray equipment 130 further comprises cameras 134 and sprayers 136, at least one of each per crop plant row. The spray equipment 130 may further include AI computer systems to read image data from the cameras 134 to identify undesirable plants, fungus infected crops, or insects and provide instructions to the sprayers 136 to spray fluid (herbicide, pesticide, fungicide, without limitation) as the sprayers 136 pass over the identified undesirable plants, fungus infected crops, or insects. The spray equipment 130 may target spray to intended areas without wasting spray on unintended areas. For example, the spray equipment 130 may target spray to weeds without wasting spray on areas where weeds are not growing.

The drones 110, as shown, are in flight over the crop 152. The drones 110 may fly in a pattern to collect image data for the entire crop field 150, or they may fly to specific locations to collect image data for the specific locations. The drones 110 may fly in a pattern to collect soil, plant, or insect diagnostic data for the entire crop field 150, or they may fly to specific locations to collect soil, plant, or insect diagnostic data for the specific locations. Soil diagnostic data may include: level of nitrogen in the soil, level of phosphorus in the soil, level of potassium in the soil, soil temperature, pH level of soil, electrical conductivity of the soil, and soil humidity or moisture level, without limitation. Plant diagnostic data may include identification of fungus, disease, and hydration, without limitation. Insect diagnostic data may comprise images or samples of insects. The drones 110 may fly in a pattern to collect soil samples for the entire field, or they may fly to specific locations to collect soil samples from the specific locations. Drones 110 may also collect samples of crop plants, leaves, insects, weeds, and soil, without limitation.

A drone launch pad 140 is shown positioned on the top of the tractor 120. The drone launch pad 140 may include: landing cites 142 for drones, batteries and battery chargers 144, imaging modules 146, and soil data tools 148, such as diagnostic testers, soil sample probes, and soil sample vacuums, without limitation. The drone launch pad 140 may include a real-time kinematic (RTK) base station.

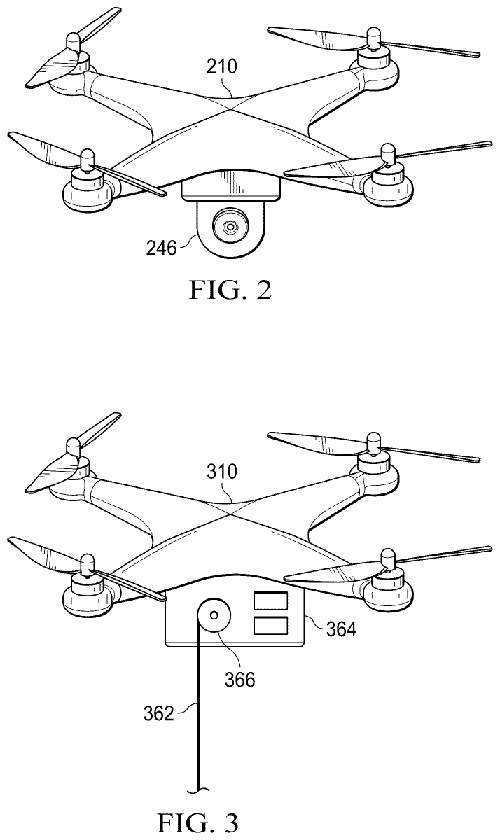

FIG. 2 shows a perspective view of a drone 210 equipped with a imaging module 246, multiple cameras, or a plurality of image sensing devices, and may provide multispectral imaging. The drone 210 may use a global navigation satellite system, such as the global position satellite (GPS) system. The drone may include global positioning (GPS) with real-time kinematic (RTK) capability, and solar sensors. The image sensing devices may provide different spectral sensitivities: ultraviolet (UV), visible spectrum (VIS), near-infrared (NIR), short-wave infrared (SWIR), and thermal, without limitation. Cameras may provide different numbers of bands per camera (mono, color, 2, 4, and 8 band multispectral), and different resolutions per band for example in the megapixel range. The image sensing devices may use light detection and ranging (LIDAR), radio detection and ranging (RADAR), and sound navigation and ranging (SONAR), without limitation.

FIG. 3 shows a perspective view of a drone 310 equipped with a line 362 that is retractable to suspend and deliver tools. The drone 310 is equipped with a tool deployment module 364 comprising a reel 366 and line 362. The tool deployment module 364 works much like a fishing reel and line in that the line 362 may be paid out when the reel 366 is allowed to rotate. The tool deployment module 364 may include a brake to stop, impede, or unimpede rotation of the reel 366 to control whether and how fast the line 362 is to be paid out. A tool (not shown) may be attached to the free end of the line 362, and the weight of the tool and gravity may assist in the line 362 being paid out. If the drone 310 pays out the line 362 until the tool is no longer suspended, but is landed on the ground, the line 362 may be taken in relatively easily by reducing the altitude of the drone 310 as the line 362 is reeled into the tool deployment module 364.

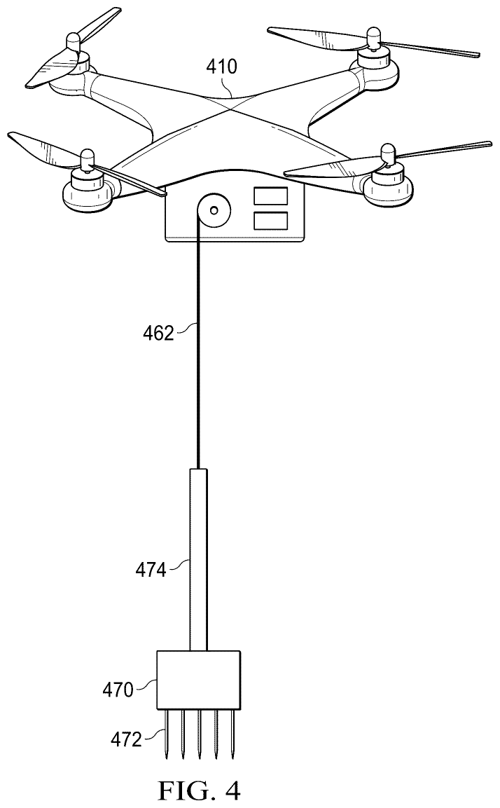

FIG. 4 shows a drone 410 equipped with a soil test probe 470 suspended by a retractable line 462. The soil test probe 470 may include one or more stabs 472 at the end of a spear 474. When the spear 474 is allowed to freefall from the drone 410, the stabs 472 may stick in the soil of the ground to a desired depth. When stuck in the soil of the ground, the soil test probe 470 may collect soil diagnostic data, which may include: level of nitrogen in the soil, level of phosphorus in the soil, level of potassium in the soil, soil temperature, pH level of soil, electrical conductivity of the soil, and soil humidity or moisture level, without limitation. The soil test probe 470 may include computer memory to store the soil diagnostic data. Alternatively, the soil diagnostic data may be transmitted to the drone 410 and/or the drone launch pad 140 (see FIG. 1). The soil diagnostic data may be transmitted to the drone 410 via transmission through the line 462 or wirelessly via radio transmission, such as BlueTooth. The soil diagnostic data may be transmitted from the soil test probe 470 or the drone 410 in real-time, or semi-real-time via wireless radio transmissions.

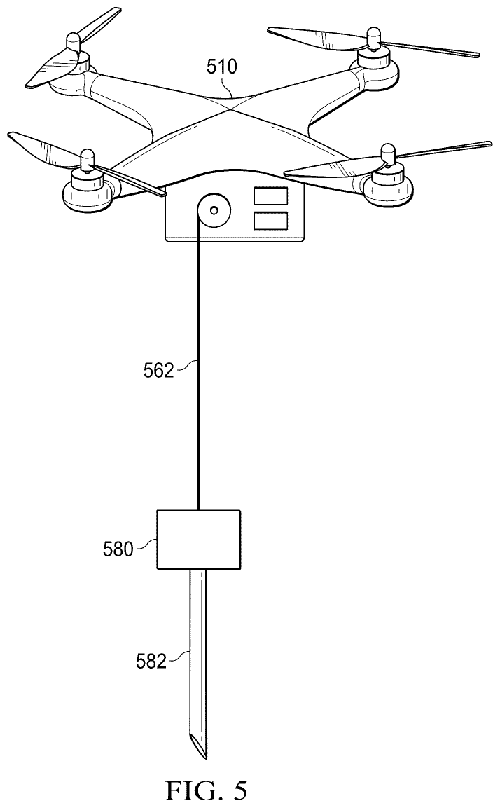

FIG. 5 shows a drone 510 equipped with a soil sample retrieval tool 580 suspended by a retractable line 562. The soil sample retrieval tool 580 may include one or more coring cylinders 582 to penetrate the soil of the ground. The soil sample retrieval tool 580 may be allowed to freefall from the drone 510, so the soil sample retrieval tool 580 may stick in the soil of the ground to a desired depth. Soil is pressed into a hollow chamber inside the coring cylinder 582. Alternatively, the tool may be a probe with a textured outer surface, so that soil particles become lodged in depressions or orifices of the textured outer surface. Alternatively, the soil sample retrieval tool 580 may comprise an auger within the coring cylinder driven by a motor to drill soil into the coring cylinder. The motor may be an electric motor or a weight that converts vertical downward movement of the weight into rotation energy to rotate the auger. The drone 510 may fly the soil sample to the drone launch pad 140 (see FIG. 1) or another location for analysis. The soil may be analyzed to determine: pH, lime content, phosphorus content, potassium content, calcium content, magnesium content, zinc content, or manganese content. The soil may be analyzed to determine:+Na, Fe, Cu, Cr, Mo, Ni, Cd, Pb+(Cation Exchange Capacity) by Mehlich I sum, Percent Base Saturation.

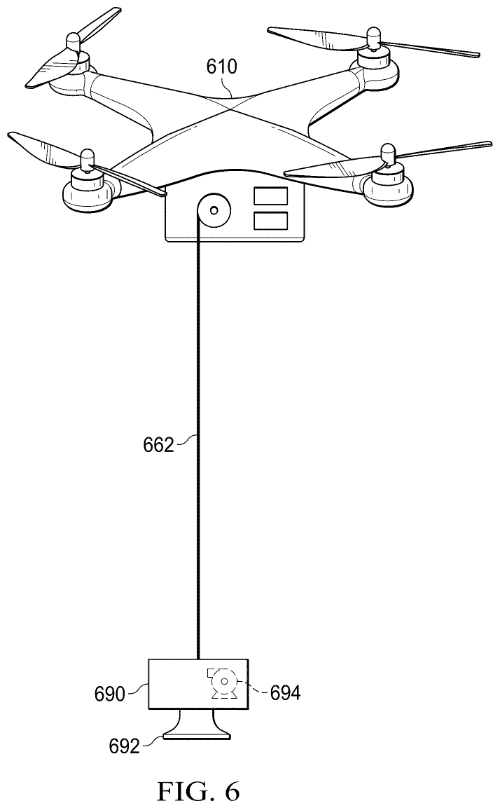

FIG. 6 shows a drone 610 equipped with a soil sample vacuum 690 suspended by a retractable line 662. The soil sample vacuum 690 may include one or more funnels 692 and a pump 694. When the soil sample vacuum 690 is landed on the ground with the opening of the funnel 692 exposed to the soil, the pump 694 may suck air and soil particles into the soil sample vacuum 690. The drone 610 may fly the soil sample to the drone launch pad 140 (see FIG. 1) or another location for analysis.

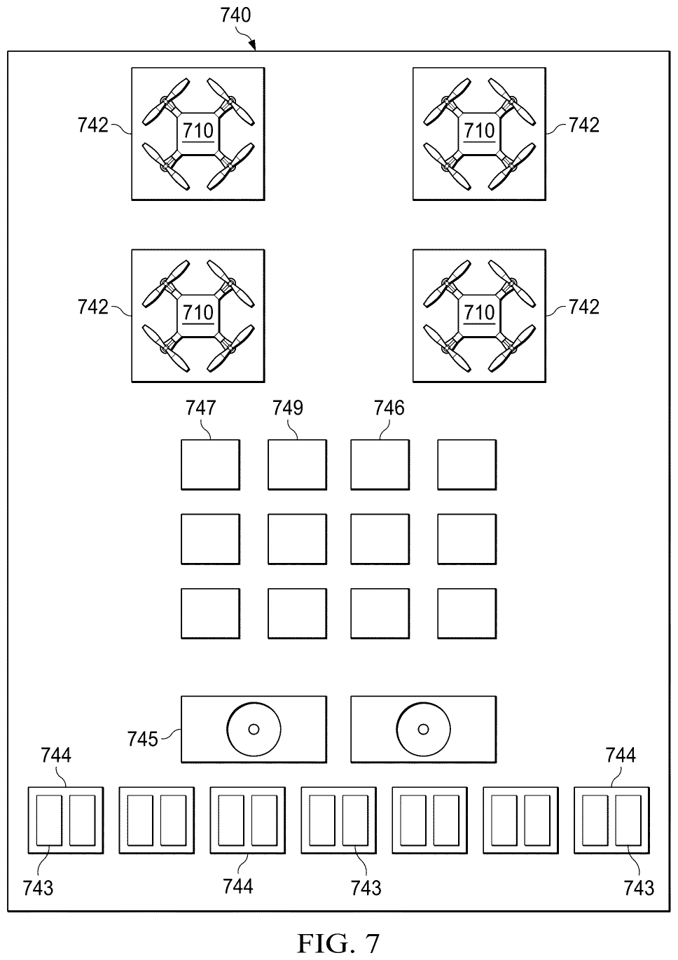

FIG. 7 shows a top view of a drone launch pad 740. The drone launch pad 740 may be attached to the top of a tractor 120 (see FIG. 1) or may be a module or skid loaded into the bed of a truck or trailer. The drone launch pad 740 may be deposited near the crop field 150 (see FIG. 1) as a stand-alone unit. The drone launch pad 740 may comprise imaging modules 746, probing modules 747, and collection modules 749, without limitation. The drone launch pad 740 may include soil sample receptacles 745, which may automatically test and analyze soil samples upon receipt. The drone launch pad 740 may also include drone batteries 743 and chargers 744. Drones 710 may automatically deposit and retrieve imaging modules 746, probing modules 747, collection modules 749, and batteries 743, without limitation. The soil sample receptacles 745 may include soil lab to automatically analyze samples and provide results to the drone launch pad 740.

A centralized warehouse may contain test and treatment kits for more obscure issues that the drones could go to pick them up from on an as needed basis. More commonly used test and treatment kits could be maintained on the drone landing pad.

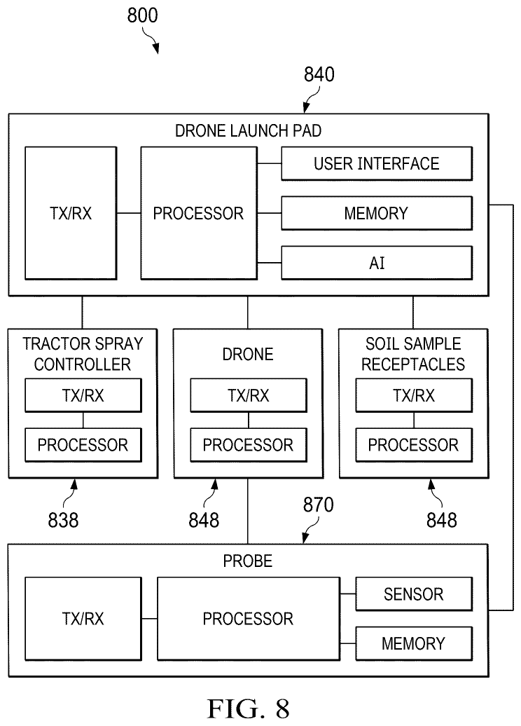

FIG. 8 shows a block diagram of a crop field diagnostic system 800. The system 800 comprises a drone launch pad 840, a tractor spray controller 838, a soil sample receptacle 848, a drone 810, and a probe 870, wherein the components have a transmitter/receiver to communicate data between the components. The drone launch pad 840 may also have a user interface, memory, and an artificial intelligence generator.

The drones may fly in a pattern to collect image data for the entire field, or they may fly to specific locations to collect image data for the specific locations. The drones may fly in a pattern to collect soil, plant, or insect diagnostic data for the entire field, or they may fly to specific locations to collect soil, plant, or insect diagnostic data for the specific locations. Soil diagnostic data may include: level of nitrogen in the soil, level of phosphorus in the soil, level of potassium in the soil, soil temperature, pH level of soil, electrical conductivity of the soil, and soil humidity or moisture level, without limitation. Plant diagnostic data may include identification of fungus, disease, and hydration, without limitation. Insect diagnostic data may comprise images or samples of insects. The drones may fly in a pattern to collect soil samples for the entire field, or they may fly to specific locations to collect soil samples from the specific locations. Drones may also collect samples of crop plants, leaves, insects, weeds, and soil, without limitation

Additionally, a robot dog could be used to collect soil samples instead of a flying drone to significantly reduce power consumption and improve the ability to do continuous operation.

Precision agriculture is enabled by automating the crop monitoring process. Spray dosages can be specific for each individual plant and customized based on the soil sample screening and other diagnostic data. The soil samples don't need to be sent back to a lab to be manually processed where it can take days, weeks or months. Samples are processed in a few minutes/hours. Soil probing data may be collected drone quicker than a human walking the field. Costs per test may be reduced by removing costs associated with collection, shipping, and analyzing the samples. Treatments (fertilizers/pesticides) may be precisely applied within minutes rather than weeks or months. This process generates higher yields and greatly reduces waste by only watering and treating where and what is desirable.

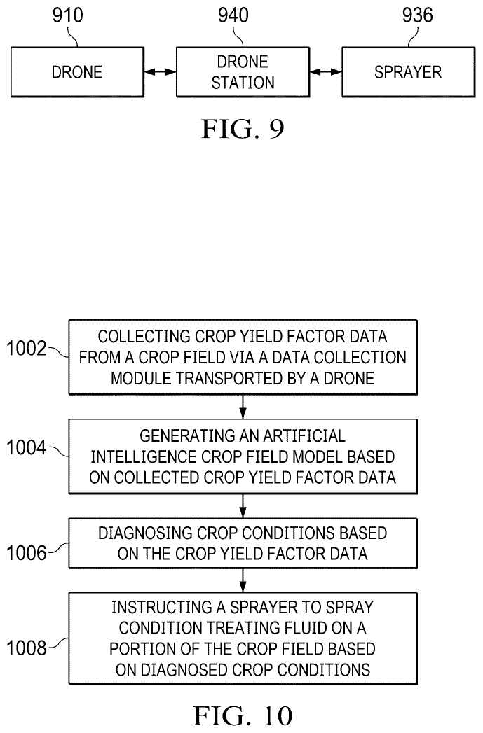

FIG. 9 shows a block diagram of a system to provide “nutritious data” to artificial intelligence engines to inform field spray. A drone 910 has a data collection module to collect crop yield factor data from a crop field. A sprayer 936 is to spray a condition treating fluid. A drone station 940 is to receive crop yield factor data from the data collection module of the drone and comprises an artificial intelligence crop field model circuit configured to: diagnose crop conditions based on the crop yield factor data; and instruct the sprayer to spray the condition treating fluid on a portion of the crop field based on diagnosed crop conditions. The artificial intelligence crop field model circuit may be implemented by instructions for execution by a processor, analog circuitry, digital circuitry, control logic, digital logic circuits programmed through hardware description language, application specific integrated circuits (ASIC), field programmable gate arrays (FPGA), programmable logic devices (PLD), or any suitable combination thereof, whether in a unitary device or spread over several devices. The artificial intelligence crop field model circuit may be implemented by instructions for execution by a processor through, for example, a function, application programming interface (API) call, script, program, compiled code, interpreted code, binary, executable, executable file, firmware, object file, container, assembly code, or object. For example, the artificial intelligence crop field model circuit may be implemented by instructions stored in a non-transitory medium such as a memory that, when loaded and executed by a processor such as a CPU (or any other suitable process), cause the functionality of the artificial intelligence crop field model circuit described herein.

FIG. 10 shows a flow chart of a method to provide “nutritious data” to artificial intelligence engines to inform field spray. Crop yield factor data is collected 1002 from a crop field via a data collection module transported by a drone. An artificial intelligence crop field model is generated 1004 based on collected crop yield factor data. Crop conditions are diagnosed 1006 based on the crop yield factor data. A sprayer is instructed 1008 to spray condition treating fluid on a portion of the crop field based on diagnosed crop conditions.

Although examples have been described above, other variations and examples may be made from this disclosure without departing from the spirit and scope of these disclosed examples.

1. A system comprising:
a drone comprising a data collection module to collect crop yield factor data from a crop field;
a sprayer to spray a condition treating fluid;
a drone station to receive crop yield factor data from the data collection module of the drone, and comprising an artificial intelligence crop field model circuit operable to:
diagnose crop conditions based on the crop yield factor data; and
instruct the sprayer to spray the condition treating fluid on a portion of the crop field based on diagnosed crop conditions.

2. The system of claim 1, wherein the data collection module comprises an image sensing device to collect crop yield factor data comprising: ultraviolet (UV) images, visible spectrum (VIS) images, near-infrared (NIR) images, short-wave infrared (SWIR) images, thermal images, light detection and ranging (LIDAR) images, radio detection and ranging (RADAR) images, or sound navigation and ranging (SONAR) images.

3. The system of claim 1, wherein the data collection module comprises a soil probe to collect crop yield factor data comprising: level of nitrogen in the soil, level of phosphorus in the soil, level of potassium in the soil, soil temperature, potential of Hydrogen (pH) level of soil, electrical conductivity of the soil, or soil humidity or moisture level.

4. The system of claim 1, wherein the data collection module comprises a soil sampler to collect crop yield factor data comprising: potential of Hydrogen (pH) of the soil, soil lime content, soil phosphorus content, soil potassium content, soil calcium content, soil magnesium content, soil zinc content, soil manganese content, soil cation exchange capacity, soil microbial activity, or soil microbiome.

5. The system of claim 1, comprising a reel connected to the drone and a line wound on the reel, wherein an end of the line is releasably connected to the data collection module, wherein the reel and line are operable to deploy and retrieve the data collection module relative to the drone.

6. The system of claim 1, wherein the sprayer is connectable to a tractor.

7. The system of claim 1, wherein the drone station comprises:
a battery charger to receive and charge a drone battery; and
a parking space to park an image sensing device, a soil probe, or a soil sampler.

8. The system of claim 1, wherein the drone station is associated with a tractor.

9. The system of claim 1, wherein the drone station comprises a soil diagnostic laboratory to analyze soil samples.

10. A method comprising:
collecting crop yield factor data from a crop field via a data collection module transported by a drone;
generating an artificial intelligence crop field model based on collected crop yield factor data;
diagnosing crop conditions based on the artificial intelligence crop field model; and
instructing a sprayer to spray condition treating fluid on a portion of the crop field based on diagnosed crop conditions.

11. The method of claim 10, wherein collecting crop yield factor data comprises collecting data via an image sensing device, the crop yield factor data comprising: ultraviolet (UV) images, visible spectrum (VIS) images, near-infrared (NIR) images, short-wave infrared (SWIR) images, thermal images, light detection and ranging (LIDAR) images, radio detection and ranging (RADAR) images, or sound navigation and ranging (SONAR) images.

12. The method of claim 10, wherein collecting crop yield factor data comprises collecting data via a soil probe, the crop yield factor data comprising: level of nitrogen in the soil, level of phosphorus in the soil, level of potassium in the soil, soil temperature, potential of Hydrogen (pH) level of soil, electrical conductivity of the soil, or soil humidity or moisture level.

13. The method of claim 10, wherein collecting crop yield factor data comprises collecting data via a soil sampler, the crop yield factor data comprising: potential of Hydrogen (pH) of the soil, soil lime content, soil phosphorus content, soil potassium content, soil calcium content, soil magnesium content, soil zinc content, soil manganese content, soil cation exchange capacity, soil microbial activity, or soil microbiome.

14. The method of claim 10, comprising:
deploying the data collection module from the drone;
collecting crop yield factor data via the data collection module; and
retrieving the data collection module to the drone.

15. The method of claim 10, comprising:
charging a drone battery via a battery charger of a drone station; and
parking the data collection module in a parking space of a drone station.

16. The method of claim 10, comprising:
transmitting data from the drone to a drone station wirelessly or via memory device.

17. The method of claim 10, comprising:
identifying global navigation satellite system coordinates of a drone station;
identifying global navigation satellite system coordinates of a data collection position within the crop field; and
navigating the drone from the drone station to the data collection position via the global navigation satellite system.

18. The method of claim 10, wherein generating an artificial intelligence crop field model comprises:
creating a crop field map with global navigation satellite system coordinates of individual crop plants; and
associating diagnosed crop conditions of individual crop plants in the crop field map.

19. The method of claim 10, wherein generating an artificial intelligence crop field model comprises:
time stamping diagnosed crop conditions.

20. The method of claim 10, wherein generating an artificial intelligence crop field model comprises:
training the artificial intelligence crop field model with the crop yield factor data in at least near real-time.

21. The method of claim 10, wherein generating an artificial intelligence crop field model comprises:
training the artificial intelligence crop field model with the crop yield factor data that corrects previous crop yield factor data.
